(19) United States
(12) Patent Application Publication
(10) Pub. No.: US 2008/0096872 A1
(43) Pub. Date: Apr. 24, 2008

(54) Composition for Treatment of Pain Specification

(76) Inventor: Robert S. Friedman, Haddonfield, NJ (US)

Correspondence Address: VENABLE LLP, P.O. BOX 34385, WASHINGTON, DC, 20043-9998, US

(21) Appl. No.: 11/794,058

(22) PCT Filed: Dec. 22, 2005
(86) PCT No.: PCT/US05/46730

§ 371 Date: Jun. 22, 2007

Related U.S. Application Data
(60) Provisional application No. 60/638,323, filed on Dec. 22, 2004.

(51) Int. Cl. A61K 31/135; A61K 31/195; A61K 31/50; A61K 31/55; A61K 31/497; A61K 31/40; A61K 31/44; A61K 31/20; A61P 19/00; A61P 25/00

(52) U.S. Cl. 514/220; 514/561; 514/646; 514/654; 514/249; 514/254.06; 514/423; 514/282; 514/560

(57) ABSTRACT

A method for the treatment of pain and/or inflammation in a subject by the administration of N-acetyl-cysteine (NAC) or derivative thereof and a pain and/or anti-inflammatory medication. The pain or anti-inflammatory medication is metabolized by the action of the cytochrome p450 system. The pain medication includes N-methyl-D-aspartate (NMDA) receptor antagonist(s). NAC and the pain medicine can be administered concurrently or sequentially. The joint administration can result in the use of lower dosages than typical dosage of the pain and/or anti-inflammatory medication or in enhanced relief from the treated condition.

### FIELD OF INVENTION

The present invention relates to methods and compositions for the treatment of pain. The composition includes a free radical scavenger active in the cytochrome p450 system and a pain medication whose primary metabolism modifiable by action of the cytochrome p450 system. In particular, the composition includes N-acetyl-cysteine (NAC) and an N-methyl-D-aspartate (NMDA) receptor antagonist.

### BACKGROUND OF THE INVENTION

Pain results from the noxious stimulation of nerve endings. Nociceptive pain is caused by noxious stimulation of nociceptors (e.g., a needle stick or skin pinch), which then transmit impulses over intact neural pathways to the spinal neurons and then to the brain. Neuropathic pain is caused by damage to neural structures, such as damage to peripheral nerve endings or nociceptors, which become extremely sensitive to stimulation and can generate impulses in the absence of stimulation (e.g., herpes zoster pain after the rash has healed). Peripheral nerve damage can lead to pathological states where there is a reduction in pain threshold (i.e., allodynia), an increased response to noxious stimuli (hyperalgesia), or an increased response duration (persistent pain). GOODMAN & GILMAN'S THE PHARMACOLOGICAL BASIS OF THERAPEUTICS 529 (Joel G. Hardman et al. eds., 9th ed. 1996); HARRISON'S PRINCIPLES OF INTERNAL MEDICINE 53-58 (Anthony S. Fauci et al. eds., 14th ed. 1998).

Neuropathic pain has been associated with a wide range of disease conditions. For instance, long-lasting allodynia has been described as a classical result of the herpes zoster (shingles) infection. Hyperalgesia has been described in AIDS patients at various stages of the disease. Burn wounds have been shown to lead to neuropathic hyperalgesia. Cancer patients receiving cytostatics and vincristine have reported experiencing hyperalgesia as a result of their chemotherapy treatment. A tumor itself can elicit hyperalgesia, perhaps as a result of chronic nerve compression by the tumor. Patients with late stage diabetes have reported hyperalgesia, often experiencing highly painful limbs with simultaneously reduced contact sensitivity of the skin. Allodynia has been reported as the diffuse pain occurring in fibromyaligia. Chronic back pain that results in compression of nerve roots of the spinal cord has also been correlated with neuropathic pain. Migraine pain has been described to include characteristic symptoms exhibited in neuropathic pain.

Recently, inhibitors of the N-methyl-D-aspartate (“NMDA”) receptors have been used to treat pain (hereinafter called “NMDA receptor antagonists”). It has been shown that NMDA receptors are involved in a wide range of processes including, neuronal death following ischemia, synaptic plasticity associated with memory formation and central sensitization during persistent pain. It is believed that glutamate, which regulates NMDA receptors, plays a key role in pain and especially chronic pain. NMDA receptors are localized throughout the central nervous system. NMDA receptors are ligand-gated cation channels that modulate sodium, potassium and calcium ions flux when they are activated by glutamate in combination with glycine. Structurally, the NMDA receptor is thought to be comprised of heteromultimeric channels containing two major subunits designated as NR1 and NR2. These subunits contain a glycine binding site, a glutamate binding site and polyamine binding site. Differential binding of these sites by NMDA antagonists can result in a variety of effects. In the CNS binding to the NR1 site can result in hallucinations and dyphoria. NR-2 binding by ketamine and by norketainine, the primary active metabolite of ketamine results in pain relief without dysphoria. The NMDA receptor also contains a magnesium (Mg++) binding site located inside the pore of the ionophore of the NMDA receptor/channel complex, which blocks the flow of ions. Phencyclidine, as well as other compounds, appear to bind to this Mg++ site. In order for PCP to gain access to the PCP receptor, the channel must first be opened by glutamate and glycine (i.e., use dependence). N-methyl-D-aspartate (“NMDA”) receptor antagonists, such as ketamine, have been used to treat postherpetic neuralgia pain, phantom limb pain, post nerve injury pain, postoperative pain, and burn pain. Furthermore, ketamine specifically oral ketamine, a prodrug for norketamine has been used in the treatment of restless legs syndrome which while not painful is characterized by uncomfortable sensations frequently seen in patients with neuropathic pain. See U.S. Pat. No. 6,855,735. Also, dextromethorphan, an NMDA receptor antagonist, metabolized to dextrorphan treats restless legs syndrome (author's data). Other NMDA receptor antagonists have been used to treat diabetic neuropathy pain and postoperative pain. Amantadine, intravenous and oral ketamine have also been used to treat pain in cancer patients.

U.S. Pat. No. 5,817,699 discloses that NMDA receptor antagonists, such as ketamine, have local-aesthetic properties and topical administration. However, the use of ketamine is also associated with harmful side-effects that curbs its clinical potential as a viable form of treatment. See U.S. Pat. No. 6,958,351. In addition, U.S. Pat. No. 5,352,683 discloses the administration of N-methyl-D-aspartate (NMDA) receptor antagonists, such as dextromethorphan, dextrorphan, as well as ketamine, for the treatment of neuropathic pain. Metabolic products of ketamine, dextromethorphan, tramadol, methadone have activity against pain. Norketamine, for example, results from the oral administration of ketamine by action of the cytochromne P450 enzyme system and is an analgesic. (Shimoyama et al., “Oral ketamine is antinociceptive in the rat formalin test: Role of the metabolite norketamine.” Pain 1999, 81: 85-93)

N-acetyl cysteine (NAC), a vitamin supplement, has been recently used for the treatment of pain and also has been shown to affect the levels of the P450 isoenzymes in animal studies. NAC has been used to treat Complex Regional Pain Syndrome Type 1 (CRPS1). See Perez et al, Pain 2003; 102:297-307. The conclusiveness of this study may be impacted by the subject's use of tramadol. The affect on P450 isoenzymes could possibly influence the metabolism, side effect profile and efficacy of medications metabolized through the P450 pathway when those medications are taken with NAC. See Chen et al. Chem. Res. Toxicol 2002; 15(7): 907-914. Specifically the effects of many drugs used for pain in addition to the NMDA receptor antagonists may be strongly influenced by the activity of the P450 system. Inhibition of the P450 system by other drugs like erythromycin has been associated with elevated serum levels of methadone and serious cardiovascular rhythm disturbances (torsade des pointes). On the other hand genetic differences in the activity of P450 has been associated with inefficacy of codeine in up to 10% of Caucasians. In this population codeine is unable to be converted to the active metabolite hydrocodone and hydromorphone. There is a need to overcome inherited and acquired deficiencies of the cytochrome P450 system to enhance the safety and efficacy of pain medication.

### SUMMARY OF THE INVENTION

The present invention relates generally to a composition and a method for the treatment of pain and inflammation. The treatment involves the co-use of N-acetyl-cysteine (NAC) or a derivative thereof and a pain medication whose primary metabolism is modifiable by action of the cytochrome p450 system. Derivatives of NAC would include salts thereof and other low carbon N-fatty acid amides. NAC is a free radical scavenger and a precursor of glutathione essential for the proper functioning of the P450 system. Pain medication, whose primary metabolism is modifiable by action of the cytochrome p450 system, include NMDA receptor antagonists such as oral ketamine, ketamine, dextromethorphan, memantine, amantadine, and meperidine. Co-use includes joint and sequential usages of NAC or derivatives and the pain medication. Such usage permits the use of compositions and kits containing these active ingredients. Mixtures of the pain medications are also possible.

The combination of NAC and oral ketamine or other NMDA receptor antagonists appears to have a synergistic effect such that the relief from pain experienced by patients is surprisingly much greater in the case of oral ketamine than would be expected from the minimal dosage of NAC used in combination with oral ketamine or by the effect of NAC by itself as a pain treatment. Moreover, similar effects are seen when NAC is combined other pain medications like tramadol, oxycodone which while not being NMDA antagonists are metabolized by the P450 system to active metabolites. The dose of ketamine is 25 mg but can range from 25 mg to 500 mg/day. The dose of NAC is 25 mg but can range from 25 to 5000 mg/day. The doses of other pain medications are those recorded in the Physician's Drug Reference (morphine, oxycodone, methadone, tramadol, NSAIDS).

Diseases to which the present invention may have application include arthritis, neurophathic pain, multiple sclerosis, restless legs syndrome, sepsis, fibromyalgia, spinal stenosis, post surgical pain, post-laminectomy pain syndrome, post-thoracotomy pain syndrome, post-mastectomy pain syndrome, somatic pain, visceral pain, conditions characterized by pain and inflammation or neuro-inflammation like neuropathic pain, complex regional pain syndrome, or neuro-inflammatory conditions characterized by distressing sensations or condition characterized by neuroinflammation of the central nervous system.

Most pain medication acts by both the primary drug and metabolites. Pain medication and other drugs can inhibit metabolism leading to excess drug accumulation and toxicity (methadone) or lack of effect (codeine, oxycodone, ketamine, tramadol). The presence of NAC affects metabolism of pain medications, improves pain relief and decreases. This is especially true in methadone treated patients. Further, the pain relieving mechanism of the present invention may also involve enhanced absorption or metabolism of oral ketamine (or other orally administered pain medications) by affecting bile acid salts that might impede absorption in the small bowel. In addition, the combination of NAC and oral ketamine (or other pain medications) might affect pain and neuroinflammation directly or by altering nuclear transcription factor (NF-Kb) or the intracellular and or intercellular messenger NO. Moreover, the presence of NAC, a precursor for glutathione is essential for the proper functioning of the P450 system necessary to the conversion of oral ketamine, dextromethorphan, methadone, oxycodone, morphine, tramadol to active pain relieving metabolites.

The amount of NAC to be used in the present invention in combination with an NMDA receptor antagonist optimally would be 25 mg but may range from 0.1 mg/kg to 10 mg/kg. The amount of the NMDA receptor antagonist can range from 5 mg to 500 mg in the composition. The ratio of NAC to 1 NMDA receptor antagonist is roughly 30:1. The ratio can vary with disease state, antagonist used and patient.

Other pain or anti-inflammatory mediations include magnesium salts, one or more steroids, non-steroid anti-inflammatory drugs, NSAIDs, essential fatty acids or fish oil products. The fatty acids include omega-3 fatty acids and metabolites. Resolvin is an active metabolite of omega-3-fatty acids made by the cyp450 system that affects inflammation and pain. (The cytochrome p450 system enzymes capable of the affecting the desired modification(s) are shown in the are shown in Table 1.) Additional pain and/or anti-inflammatory mediations include morphine, methadone, oxycodone, hydromorphone, codeine, fentanyl or mixtures thereof and also oxycodone, prozac, dyazide, zyprexa, neurontin, trazodone, paxil or mixtures thereof.

The pain or anti-inflammatory medication can be formulated in time release, extended release, controlled release or sustained release forms.

NAC or derivatives thereof and the pain or anti-inflammatory medication can be administered separately, contemporaneously or sequentially. Convention modes of administration are envisioned.

The use of the compositions of the invention also can results in a lesser than normal dosage amount of pain or inflammation medication ascribed for treatment of the condition or decreases the presence of metabolic products with toxicity.

The components of the inventive composition can be packaged as a kit. The NAC or derivatives thereof and the pain or anti-inflammatory medication can be placed in separate containers or in a single container where their relative proportions are selected for prophylaxis or treatment of s specified condition. Any type of container or sub-package can be selected. Multiple medications can be selected. A series of single dosage forms can be selected. The kit may include additional materials which would facilitate or be deemed necessary for prophylaxis or treatment of a condition and may be assemble to effect a regimen. The kits may contain items to facilitate the use, e.g. instructions, containers, test tubes, etc.

### DETAILED DESCRIPTION OF THE INVENTION

NAC affects the cyp450 system (P450), a family of 20 enzyme families defined by homologies with 40% of their DNA sequence. These families include 1A2; 2B6; 2C8; 2C9; 2E1; and 3A4,5,7. Isozymes most important in drug metabolism are cyp1a2, cyp2d6, cyp2c9, cyp2c19, and cyp3a3/4. Methadone, ketamine, dextromethorphan (and possibly other pain medications) are specifically important in that they inhibit their own metabolism which the invention reverses with NAC. Other drugs like the tricyclic antidepressants can inhibit the cyp450(P450) system but some are not reversible with NAC like amitriptyline. Genetic polymorphism can reduce the activity of these enzymes as well making certain populations of patients more susceptible to inhibition. In white populations 10% are poor metabolizers. In West Africans, the incidence is as high as 18%. Steroids, carbamazepine, phenobarbital can induce these enzymes. While older drugs like cimetidine inhibit these enzymes newer compounds are being developed that specifically don't affect this system (pregabalin for pain). Stress (oxidative, disease, aging, cancer, organ failure) acts to reduce the activity of this system-putting patients at risk. (see Bernard, J. Clin Oncology; Vol. 8 (2000), pp. 1780-1812). Note also with respect to pain, independent of the cyp system-NAC's effect is specifically glutathione dependent. ((see Wagner, R. et al., “Wallerian degeneration and hyperalgesia after peripheral nerve injury are glutathione dependent,” Pain, Vol. 77 (1998), pp. 173-179). Therefore NAC/glutathione could act either at the site of tissue injury or by promoting the synthesis of active metabolites to provide pain relief. While little is known about NAC acting as a precursor for glutathione affects the activity of the cyp 450 enzymes so important in the metabolism of analgesic compounds like methadone and ketamine. Norketamine is the metabolite cyp450 dependent for the NMDA drug ketamine.

The specific activity of NAC is that it seems to be responsible for protecting certain enzymes in the family of the cytochrome system responsible for creating active metabolites of pain killing drugs specifically ketamine, methadone, tramadol, oxycodone, and possibly morphine. NAC acts as an antioxidant specifically in these instances to reverse the inactivation of cyp2d6,cyp3a4 either by the drugs themselves, by depletion of glutathione (an amino acid responsible for the overall “health” of the cyp 450 system) or by other commonly used drugs used in pain like the SSRI's-paroxetine, sertaline, but not nortriptyline whose inhibition of cyp3a4 is NAC resistant.

Genetics, substrates, inhibitors and inducers of typical enzymes in the P450 system are shown in Table 1 many of which could be affected by NAC.

TABLE 1

| | | | | | | | |
|---|---|---|---|---|---|---|---|
| 1A2 | 2B6 | 2C8 | 2C19 | 2C9 | 2D6 | 2E1 | 3A4,5,7 |

Substrates

| | | | | | | | |
|---|---|---|---|---|---|---|---|
| amitriptyline | bupropion | paclitaxel | Proton Pump | NSAIDs: | Beta Blockers: | Anesthetics: | Macrolide |
| caffeine | cyclophosphamide | torsemide | Inhibitors: | diclofenac | carvedilol | enflurane | antibiotics: |
| clomipramine | efavirenz | amodiaquine | lansoprazole | ibuprofen | S-metoprolol | halothane | clarithromycin |
| clozapine | ifosfamide | cerivastatin | omeprazole | S- | propafenone | isoflurane | erythromycin |
| cyclobenzaprine | methadone | repaglinide | pantoprazole | naproxen=>Nor | timolol | methoxyflurane | (not 3A5) |
| estradiol | | | E-3810 | piroxicam | Antidepressants: | sevoflurane | NOT |
| fluvoxamine | | | Anti-epileptics: | suprofen | amitriptyline | acetaminophen | azithromycin |
| haloperidol | | | diazepam=>Nor | Oral | clomipramine | =>NAPQI | telithromycin |
| imipramine N- | | | phenytoin(O) | Hypoglycemic | desipramine | aniline | Anti- |
| DeMe | | | S-mephenytoin | Agents: | imipramine | benzene | arrhythmics: |
| mexilletine | | | phenobarbitone | tolbutamide | paroxetine | chlorzoxazone | quinidine=>3- |
| naproxen | | | amitriptyline | glipizide | Antipsychotics: | ethanol | OH (not 3A5) |
| olanzapine | | | carisoprodol | Angiotensin II | haloperidol | N,N-dimethyl | Benzodiazepines: |
| ondansetron | | | citalopram | Blockers: | perphenazine | formamide | alprazolam |
| phenacetin=> | | | clomipramine | losartan | risperidone=>9 | theophylline | diazepam=>3OH |
| acetaminophen | | | cyclophosphamide | irbesartan | OH | =>8-OH | midazolam |
| =>NAPQI | | | hexobarbital | Sulfonylureas: | thioridazine | | triazolam |
| propranolol | | | imipramine N- | glyburide/ | alprenolol | | Immune |
| riluzole | | | DeME | glibenclamide | amphetamine | | Modulators: |
| ropivacaine | | | indomethacin | glipizide | aripiprazole | | cyclosporine |
| tacrine | | | R- | glimepiride | atomoxetine | | tacrolimus |
| theophylline | | | mephobarbital | tolbutamide | bufuralol | | (FK506) |
| tizanidine | | | moclobemide | amitriptyline | chlorpheniramine | | HIV Antivirals: |
| verapamil | | | nelfinavir | celecoxib | chlorpromazine | | indinavir |
| (R)warfarin | | | nilutamide | fluoxetine | codeine (=>O- | | nelfinavir |
| zileuton | | | primidone | fluvastatin | desMe) | | ritonavir |
| zolmitriptan | | | progesterone | glyburide | debrisoquine | | saquinavir |
| | | | proguanil | nateglinide | dexfenfluramine | | Prokinetic: |
| | | | propranolol | phenytoin=>4- | dextromethorphan | | cisapride |
| | | | teniposide | OH | duloxetine | | Antihistamines: |
| | | | R-warfarin=>8- | rosiglitazone | encainide | | astemizole |
| | | | OH | tamoxifen | flecainide | | chlorpheniramine |
| | | | | torsemide | fluoxetine | | terfenidine |
| | | | | S-warfarin | fluvoxamine | | Calcium |
| | | | | | lidocaine | | Channel |
| | | | | | metoclopramide | | Blockers: |
| | | | | | methoxy- | | amlodipine |
| | | | | | amphetamine | | diltiazem |
| | | | | | mexilletine | | felodipine |
| | | | | | minaprine | | lercanidipine |
| | | | | | nebivolol | | nifedipine |
| | | | | | nortriptyline | | nisoldipine |
| | | | | | ondansetron | | nitrendipine |
| | | | | | perhexiline | | verapamil |
| | | | | | phenacetin | | HMG CoA |
| | | | | | phenformin | | Reductase |
| | | | | | propranolol | | Inhibitors: |
| | | | | | sparteine | | atorvastatin |
| | | | | | tamoxifen | | cerivastatin |
| | | | | | tramadol | | lovastatin |
| | | | | | venlafaxine | | NOT pravastatin |
| | | | | | | | simvastatin |
| | | | | | | | Steroid 6beta- |
| | | | | | | | OH: |
| | | | | | | | estradiol |
| | | | | | | | hydrocortisone |
| | | | | | | | progesterone |
| | | | | | | | testosterone |
| | | | | | | | Miscellaneous: |
| | | | | | | | alfentanyl |
| | | | | | | | aripiprazole |
| | | | | | | | buspirone |
| | | | | | | | cafergot |
| | | | | | | | caffeine=>TMU |
| | | | | | | | cilostazol |
| | | | | | | | cocaine |
| | | | | | | | codeine-N- |
| | | | | | | | demethylation |
| | | | | | | | dapsone |
| | | | | | | | dextromethorphan |
| | | | | | | | docetaxel |
| | | | | | | | domperidone |
| | | | | | | | eplerenone |
| | | | | | | | fentanyl |
| | | | | | | | finasteride |
| | | | | | | | gleevec |
| | | | | | | | haloperidol |
| | | | | | | | irinotecan |
| | | | | | | | LAAM |
| | | | | | | | lidocaine |
| | | | | | | | methadone |
| | | | | | | | nateglinide |
| | | | | | | | odanestron |
| | | | | | | | pimozide |
| | | | | | | | propranolol |
| | | | | | | | quinine |
| | | | | | | | NOT |
| | | | | | | | rosuvastatin |
| | | | | | | | salmeterol |
| | | | | | | | sildenafil |
| | | | | | | | sirolimus |
| | | | | | | | tamoxifen |
| | | | | | | | taxol |
| | | | | | | | terfenadine |
| | | | | | | | trazodone |
| | | | | | | | vincristine |
| | | | | | | | zaleplon |
| | | | | | | | zolpidem |

Inhibitors

| | | | | | | | |
|---|---|---|---|---|---|---|---|
| amiodarone | thiotepa | trimethoprim | chloramphenicol | amiodarone | amiodarone | diethyl- | HIV Antivirals: |
| cimetidine | ticlopidine | quercetin | cimetidine | fluconazole | buproprion | dithio- | delaviridine |
| fluoroquinolones | | glitazones | felbamate | fluvastatin | celecoxib | carbamate | indinavir |
| fluvoxamine | | gemfibrozil | fluoxetine | fluvoxamine | chlorpromazine | disulfiram | nelfinavir |
| furafylline | | montelukast | fluvoxamine | isoniazid | chlorpheniramine | | ritonavir |
| interferon? | | | indomethacin | lovastatin | cimetidine | | amiodarone |
| methoxsalen | | | ketoconazole | phenylbutazone | citalopram | | aprepitant |
| mibefradil | | | lansoprazole | probenicid | clomipramine | | NOT |
| | | | modafinil | sertraline | cocaine | | azithromycin |
| | | | omeprazole | sulfamethoxazole | doxepin | | chloramphenicol |
| | | | oxcarbazepine | sulfaphenazole | doxorubicin | | cimetidine |
| | | | probenicid | teniposide | duloxetine | | ciprofloxacin |
| | | | ticlopidine | trimethoprim | escitalopram | | clarithromycin |
| | | | topiramate | zafirlukast | fluoxetine | | diethyl- |
| | | | | | halofantrine | | dithiocarbamate |
| | | | | | red-haloperidol | | diltiazem |
| | | | | | levomepromazine | | erythromycin |
| | | | | | metoclopramide | | fluconazole |
| | | | | | methadone | | fluvoxamine |
| | | | | | mibefradil | | gestodene |
| | | | | | moclobemide | | grapefruit juice |
| | | | | | paroxetine | | itraconazole |
| | | | | | quinidine | | ketoconazole |
| | | | | | ranitidine | | mifepristone |
| | | | | | ritonavir | | nefazodone |
| | | | | | sertraline | | norfloxacin |
| | | | | | terbinafine | | norfluoxetine |
| | | | | | ticlopidine | | mibefradil |
| | | | | | histamine H1 | | star fruit |
| | | | | | receptor | | verapamil |
| | | | | | antagonists | | |
| | | | | | diphenhydramine | | |
| | | | | | chlorpheniramine | | |
| | | | | | clemastine | | |
| | | | | | perphenazine | | |
| | | | | | hydroxyzine | | |
| | | | | | tripelennamine | | |

Inducers

| | | | | | | | |
|---|---|---|---|---|---|---|---|
| broccoli | phenobarbital | rifampin | carbamazepine | rifampin | dexamethasone | ethanol | HIV Antivirals: |
| brussel sprouts | http:// | | norethindrone | secobarbital | rifampin | isoniazid | efavirenz |
| char-grilled | medicine.- | | NOT | | | | nevirapine |
| meat | iupui.edu/- | | pentobarbital | | | | barbiturates |
| insulin | flockhart/- | | prednisone | | | | carbamazepine |
| methyl | 2B6.htm- | | rifampin | | | | glucocorticoids |
| cholanthrene | 2B6- | | | | | | modafinil |
| modafinil | phenytoinrifampin | | | | | | phenobarbital |
| nafcillin | | | | | | | phenytoin |
| beta- | | | | | | | rifampin |
| naphthoflavone | | | | | | | St. John's wort |
| omeprazole | | | | | | | troglitazone |
| tobacco | | | | | | | oxcarbazepine |
| | | | | | | | pioglitazone |
| | | | | | | | rifabutin |

Genetics

| | | | | | | | |
|---|---|---|---|---|---|---|---|
| Chromosome | Chromosome | Chromosome | Chromosome | Chromosome | Chromosome | Chromosome | Chromosome 7 |
| 15 | 19 | 10 | 10 | 10 | 22 | 10 | |
| N/A | Polymorphic | | Polymorphic | Polymorphic | Polymorphic | N/A | N/A |
| N/A | 3-4% | | 3-5% Caucasian | 1-3% Caucasian | 5-10% | N/A | N/A |
| | Caucasians | | PMs, 15-20% | PMs | Caucasian PMs | | |
| | PMs | | Asian PMs | | | | |

The pain-alleviating compounds or compositions, including neuropathic pain-alleviating compounds or compositions, presented herein may be compounded, using conventional methodologies, for example, with the usual non-toxic, pharmaceutically acceptable excipients, carriers, diluents or other adjuvants. The choice of adjuvants will depend upon the active ingredients employed, the physical form of the composition, the route of administration, and other factors. Routes of administration may include oral, intravenous, intrathecal or topical, preferably oral.

The excipients, binders, carriers, and diluents which can be used include water, glucose, lactose, natural sugars such as sucrose, glucose, or corn sweeteners, sorbitol, natural and synthetic gums such as gum acacia, tragacanth, sodium alginate, and gum arabic, gelatin, mannitol, starches such as starch paste, corn starch, or potato starch, magnesium trisilicate, talc, keratin, colloidal silica, urea, stearic acid, magnesium stearate, dibasic calcium phosphate, crystalline cellulose, methyl cellulose, carboxymethyl cellulose, polyethylene glycol, waxes, glycerin, and saline solution, among others.

Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin. The dosage forms can also comprise one or more acidifying agents, adsorbents, alkalizing agents, antiadherents, antioxidants, binders, buffering agents, colorants, complexing agents, diluents or fillers, direct compression excipients, disintegrants, flavorants, fragrances, glidants, lubricants, opaquants, plasticizers, polishing agents, preservatives, sweetening agents, or other ingredients known for use in pharmaceutical preparations.

Antiadherent are agents that prevents the sticking of solid dosage formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

Antioxidants are agents which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate and sodium metabisulfite and other materials known to one of ordinary skill in the art.

Binders are substances used to cause adhesion of powder particles in solid dosage formulations. Such compounds include, by way of example and without limitation, acacia, alginic acid, carboxymethylcellulose sodium, poly(vinylpyrrolidone), compressible sugar (e.g., NuTab), ethylcellulose, gelatin, liquid glucose, methylcellulose, povidone and pregelatinized starch and other materials known to one of ordinary skill in the art.

When needed, binders may also be included in the dosage forms. Exemplary binders include acacia, tragacanth, gelatin, starch, cellulose materials such as methyl cellulose, HPMC, HPC, HEC and sodium carboxy methyl cellulose, alginic acids and salts thereof, polyethylene glycol, guar gum, polysaccharide, bentonites, sugars, invert sugars, poloxamers (PLURONIC™ F68, PLURONIC™ F127), collagen, albumin, gelatin, cellulosics in nonaqueous solvents, combinations thereof and others known to those skilled in the art. Other binders include, for example, polypropylene glycol, polyoxyethylene-polypropylene copolymer, polyethylene ester, polyethylene sorbitan ester, polyethylene oxide, combinations thereof and other materials known to one of ordinary skill in the art.

Buffering agents are compounds used to resist changes in pH upon dilution or addition of acid or alkali. Such compounds include, by way of example and without limitation, potassium metaphosphate, potassium phosphate, monobasic sodium acetate and sodium citrate anhydrous and dihydrate and other materials known to one of ordinary skill in the art. Sweetening agents are compounds used to impart sweetness to a preparation. Such compounds include, by way of example and without limitation, aspartame, dextrose, glycerin, mannitol, saccharin sodium, sorbitol, sucrose, and other materials known to one of ordinary skill in the art.

Diluents or fillers are inert substances used to create the desired bulk, flow properties, and compression characteristics in the preparation of solid dosage forms. Such compounds include, by way of example and without limitation, dibasic calcium phosphate, kaolin, lactose, dextrose, magnesium carbonate, sucrose, mannitol, microcrystalline cellulose, powdered cellulose, precipitated calcium carbonate, calcium sulfate, sorbitol, and starch and other materials known to one of ordinary skill in the art.

Direct compression excipients are compounds used in compressed solid dosage forms. Such compounds include, by way of example and without limitation, dibasic calcium phosphate (e.g., Ditab) and other materials known to one of ordinary skill in the art.

Disintegrants are compounds used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starches thereof, sweeteners, clays such as bentonite, microcrystalline cellulose (e.g., Avicel), methyl cellulose, carboxymethylcellulose calcium, sodium carboxymethylcellulose, alginic acid, sodium alginate, cellulose polyacrilin potassium (e.g., Amberlite), alginates, sodium starch glycolate, gums, agar, guar, locust bean, karaya, xanthan, pectin, tragacanth, agar, bentonite, and other materials known to one of ordinary skill in the art.

Glidants are agents used in solid dosage formulations to promote flowability of the solid mass. Such compounds include, by way of example and without limitation, colloidal silica, cornstarch, talc, calcium silicate, magnesium silicate, colloidal silicon, tribasic calcium phosphate, silicon hydrogel and other materials known to one of ordinary skill in the art. Lubricants are substances used in solid dosage formulations to reduce friction during compression. Such compounds include, by way of example and without limitation, sodium oleate, sodium stearate, calcium stearate, zinc stearate, magnesium stearate, polyethylene glycol, talc, mineral oil, stearic acid, sodium benzoate, sodium acetate, sodium chloride, and other materials known to one of ordinary skill in the art.

Colorants are compounds used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, ferric oxide, other FD&C dyes and natural coloring agents such as grape skin extract, beet red powder, beta-carotene, annato, carmine, turmeric, paprika, and other materials known to one of ordinary skill in the art. The amount of coloring agent used will vary as desired.

Flavorants are compounds used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those skilled in the art. Particularly contemplated flavors are the grape and cherry flavors and citrus flavors such as orange.

Exemplary preservatives include materials that inhibit bacterial growth, such as Nipagin, Nipasol, alcohol, antimicrobial agents, benzoic acid, sodium benzoate, benzyl alcohol, sorbic acid, parabens, isopropyl alcohol and others known to one of ordinary skill in the art. Solid dosage forms of the invention can also employ one or more surface active agents or cosolvents that improve wetting or disintegration of the core and/or layer of the solid dosage form.

Solid dosage forms of the invention can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

A water soluble coat or layer can be formed to surround a solid dosage form or a portion thereof. The water soluble coat or layer can either be inert or drug-containing. Such a coat or layer will generally comprise an inert and non-toxic material which is at least partially, and optionally substantially completely, soluble or erodible in an environment of use. Selection of suitable materials will depend upon the desired behavior of the dosage form. A rapidly dissolving coat or layer will be soluble in the buccal cavity and/or upper GI tract, such as the stomach, duodenum, jejunum or upper small intestines. Exemplary materials are disclosed in U.S. Pat. No. 4,576,604 to Guittard et al. and U.S. Pat. No. 4,673,405 to Guittard et al., and U.S. Pat. No. 6,004,582 to Faour et al. and the text Pharmaceutical Dosage Forms: Tablets Volume I, 2.sup.nd Edition. (A. Lieberman. ed. 1989, Marcel Dekker, Inc.), the disclosures of which are hereby incorporated by reference. In some embodiments, the rapidly dissolving coat or layer will be soluble in saliva, gastric juices, or acidic fluids.

For transcutaneous or transdermal administration, the compounds may be combined with skin penetration enhancers such as propylene glycol, polyethylene glycol, isopropanol, ethanol, oleic acid, N-methylpyrrolidone, or others known to those skilled in the art, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, or others known to those skilled in the art, to provide the composition in gel form, which can be dissolved in solvent such as methylene chloride, evaporated to the desired viscosity, and then applied to backing material to provide a patch.

For intravenous, intramuscular, subcutaneous, intrathecal, epidural, perineural or intradermal administration, the active ingredients may be combined with a sterile aqueous solution. The solution may be isotonic with the blood of the recipient. Such formulations may be prepared by dissolving one or more solid active ingredients in water containing physiologically compatible substances such as sodium chloride, glycine, or others known to those skilled in the art, and/or having a buffered pH compatible with physiological conditions to produce an aqueous solution, and/or rendering the solution sterile. The formulations may be present in unit dose containers such as sealed ampoules or vials.

For topical (e.g., dermal or subdermal) or depot administration, the active ingredients may be formulated with oils such as cottonseed, hydrogenated castor oil and mineral oil; short chain alcohols as chlorobutanol and benzyl alcohol; also including polyethylene glycols, polysorbates; polymers such as sucrose acetate isobutyrate, caboxymethocellusose and acrylates; buffers such as dihydrogen phosphate; salts such as sodium chloride and calcium phosphate; and other ingredients included but not exclusive to povidone, lactose monohydrate, magnesium stearate, myristyo-gamma-picolinium; and water.

A solid dosage form of the invention can be coated with a finish coat as is commonly done in the art to provide the desired shine, color, taste or other aesthetic characteristics. Materials suitable for preparing the finish coat are well known in the art and found in the disclosures of many of the references cited and incorporated by reference herein.

Various other components, in some cases not otherwise listed above, can be added to the present formulation for optimization of a desired active agent release profile including, by way of example and without limitation, glycerylmonostearate, nylon, cellulose acetate butyrate, d,l-poly(lactic acid), 1,6-hexanediamine, diethylenetriamine, starches, derivatized starches, acetylated monoglycerides, gelatin coacervates, poly(styrene-maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

The present, pain-alleviating compositions, including neuropathic pain-alleviating compositions, can be formulated in capsules, tablets, caplets, or pills. Such capsules, tablets, caplets, or pills of the present neuropathic pain-alleviating compositions can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Similarly, the carrier or diluent may include any sustained release material known in the art, such as glyceryl monostearate or glyceryl distearate, alone or mixed with a wax. The formulations of the invention may be formulated so as to provide quick, sustained, or delayed release of the active ingredient after administration to the patient by employing procedures well known in the art.

Controlled release or sustained-release dosage forms, as well as immediate release dosage forms are specifically contemplated. Controlled release or sustained release as well as immediate release compositions in liquid forms in which a therapeutic agent may be incorporated for administration orally or by injection are also contemplated. Control release forms may also have the advantage of favoring reverse isomerization, that is the preferential conversion of the parent compound to its more active and less toxic form like s-norketamine from oral ketamine.

Pain-alleviating compositions, including neuropathic pain-alleviating compositions, presented herein can be administered from about one time daily to about six times daily, two times daily to about four times daily, or one time daily to about two times daily.

Pain-alleviating compositions, including neuropathic pain-alleviating compositions, presented herein preferably comprise at least one colloidal dispersion system, additive or preservative, diluent, binder, plasticizer, or slow release agent.

It should be understood that compounds used in the art of pharmaceutical formulation generally serve a variety of functions or purposes. Thus, whether a compound named herein is mentioned only once or is used to define more than one term herein, its purpose or function should not be construed as being limited solely to the named purpose(s) or function(s). The present pain-alleviating compounds or compositions, including neuropathic pain-alleviating compounds or compositions, may be in admixture with an organic or inorganic carrier or excipient suitable for administration in enteral or parenteral applications, such as orally, topically, transdermally, by inhalation spray, rectally, by subcutaneous, intravenous, intramuscular, subcutaneous, intrathecal, epidural, perineural, intradermal, intraocular injection or infusion techniques. Preferably, such compositions are in the form of a topical, intravenous, intrathecal, epidural, perineural, or oral formulation. More preferably, such compositions are in the form of an intrathecal, epidural or perineural formulation. Even more preferably, such compositions are in the form of an intravenous formulation. Most preferably, such compositions are in the form of an oral formulation.

The study, which follows, relates to 6 subjects who, while taking NAC and methadone for chronic pain, seem to be more tolerant of methadone's sedative side effects. In this study, the serum levels of methadone and its metabolites were evaluated in these subjects to see if there were differences in the serum levels of methadone and methadone metabolites in the presence and absence of NAC. The Institutional Review Board approved protocol used is as follows:

Protocol

Research Design and Methods

Clinical Study Site:

Clinical offices of Burlington Anesthesia associates, an affiliate of Virtua Health, Mt. Holly, N.J.

Target Population

6 patients who have been on chronic methadone therapy for pain treatment and who are also taking NAC (600 mg/day) were studied. Patients were taking a range of methadone doses (30-160 mg/day).

Inclusion Criteria:

Severe chronic pain requiring methadone treatment

Exclusion Criteria:

Refusal to provide consent for a blood test.

Allergy to sulfa compounds

Research Design

This is a descriptive case study of 6 patients. A blood sample was obtained and used to measure serum methadone levels was obtained on methadone/NAC therapy. NAC was supplied by the investigator from assayed commercially available supply and pill counts were done to assure patient compliance. The patients were on the same methadone dose for over seven days; this ensures steady state levels of methadone had been achieved. The patients were then be asked to stop taking NAC for 7 days and were asked to return to the clinic for a second blood draw that measured serum methadone levels. Blood was collected in the offices of BAA. Three additional patients were studied, after the results from sera of the initial patients were assayed; these patients had baseline pain and fatigue assessments with the Piper Survey and were subsequently treated with NAC, serum determinations were sent, but have not as yet been completed. Thus patients on methadone were studied with NAC withdrawal (First Group) and NAC added to treatment (second group). The medical chart was reviewed for current medications, including over the counter medications, supplements, and herbal preparations; health conditions and available laboratory evaluations of electrolytes. The patient's age and gender was recorded.

Procedures

BAA offices: A 5 ml blood sample will be obtained on methadone/NAC. Blood was drawn prior to a normally timed dose of methadone. The patient was instructed to discontinue taking NAC and returned in 7 days, at the same time, for a blood draw. This was also drawn prior to a normally timed dose of methadone. Patients completed a Piper survey for fatigue and distress on each occasion prior to the blood draw. The Piper score reflects a valid measure of fatigue. Higher numbers for the score are indicative of greater fatigue. Within the Piper survey a pain measure is also recorded. Both pain and fatigue scores were measured at each of the clinical visits one week apart. Trained study personnel completed chart review. Analysis of serum concentrations of methadone was completed at the Bioanalytical Core Lab at the Arizona Health Science Center (Sarver Heart Center, University of Arizona, Tucson).

Serum assay: Approximately 5 mL of blood was collected at baseline and after three days off NAC. The resulting serum samples were analyzed for the separate enantiomers of methadone and its two major metabolites, 2-ethylidene-1,5-dimethyl-3,3-biphenylpyrrolidine (EDDP). Sample preparation was by solid phase extraction (SPE) using 200 mg Bond Elut C₁₈ cartridges (Varian, USA) (See Boulton D W FAU, DeVane C L. Chirality 2000; 12(9):681-687.) while the chromatography was performed on a Series 1100 LC/MS equipped with an atmospheric pressure ionization electrospray detector (AP-ESI), (Agilent Technologies, USA) using a CYCLOBOND® 12000 RSP chiral column (ASTEC, USA).

The samples was shipped to the Bioanalytical Core Lab identified with only a number to ensure patient confidentiality.

Clinical Samples

The data summary follows from the protocols taught above. The key finding is that in all cases the ratio of metabolite/parent isomer (rmtd/reddp; smtd/seddp) is lower in each patient when NAC is used—the trend is consistent even though the differences in ratios may not be statistically significant. In patient 3, where paxil a potent inhibitor of the cytp450 system, is taken the effect of NAC is even more pronounced.

Improvement in fatigue and reduction in medication for pain were seen when NAC is added to methadone in the cases summarized and a few additional cases. The mechanism may be that NAC shifts metabolism from a major inactive metabolite to a minor clinically active metabolite for methadone-methadol; and may possibly do the same for the generation of the norketamine metabolite from ketamine in those subjects who are treated with that medication. Studies on the effect of liver disease on the fecal excretion of major (EDDP) as well as minor products of methadone metabolism (Kreek, M. J., “Effects of liver disease on fecal excretions of methadone and its unconjugated metabolites in maintenance patients,” Biomed. Mass Spectrometry 1983: 10: 544-549)) suggest that in female patients with liver disease alterations in methadone metabolism resulting in a decrease in the metabolism of methadone and decrease levels of the inactive metabolite, EDDP, but decrease the levels of the active metabolite, methadol even more.

Clinical samples from pain patients receiving NAC either as primary or add-on treatment with pain medication were analyzed for the (R) and (S) enantiomers of both methadone (MTD) and its major metabolite, (±)-2-ethyl-1,5-dimethyl-3,3-diphenylpyrrolinium perchlorate (EDDP) by a sterioselective LC/MS method which had been previously developed and validated in this laboratory. The calibration curves for the separate enantiomers, R— and S-MTD, are linear from 15.6 ng mL⁻¹ to 400 ng mL⁻¹ with a correlation coefficient (r²) of >0.996 from a 100 uL sample. Similarly, the curves for the separate enantiomers of EDDP are linear from 15.6 ng mL⁻¹ to 500 ng mL⁻¹ with a correlation coefficient (r²) of >0.996. Liquid-liquid extraction was used for sample preparation. A portion of the aqueous extract was then introduced onto the chromatographic system for analysis. Quality control samples, consisting of spiked plasma at three levels, LQC (25 ng mL⁻¹ per enantiomers), MQC (100 ng mL⁻¹ per enantiomers) and HQC (400 ng mL⁻¹ per enantiomers), in triplicate, were included with each set of samples and calibration standards.

The instrumentation used consisted of a Series 1100 LC/MSD (Agilent Technologies, USA) equipped with a mass selective detector (MSD) supplied with atmospheric pressure ionization electrospray (API-ES). The MSD was set for selective ion monitoring (SIM) at 310 m/z (MTD) and 278 m/z (EDDP). For data collection and automated sample analysis, the system was interfaced to a Kayak XA computer (Hewlett-Packard, USA) running ChemStation software (Agilent Technologies, USA). Variability of samples with quality control was less than 1%.

The following is a summary of additional patients treated with NAC who had been treated for a variety of painful conditions with NMDA receptor antagonists and analgesics metabolized by Cyp 450.

TABLE 2
Clinical Data on Patients Treated with NAC

| Patient No | | Condition | | Treatment | Observations/Comments |
|---|---|---|---|---|---|
| 1 | GS | Failed | Restless | NAC/ketamine/ | Better |
| | | laminecotomy | Legs | methadone | |
| | | | syndrome | | |
| | | | (RLS)/ | | |
| | | | pain | | |
| 2 | PR | Spinal/ | RLS/ | NAC/ketamine/ | Better |
| | | stenosis | pain | mo | |
| 3 | RL | Peripheral | pain | NAC/ketamine/ | better |
| | | Nerve injury | | methadone | combo-new form-no relief |
| 4 | LC | Failed | RLS | NAC/ketamine/ | Better |
| | | Laminecotomy | | tramadol | |
| 5 | CD | Failed | | NAC/ketamine/ | No better |
| | | laminecotomy/ | | methadone | ?compliance |
| | | pain | | | |
| 6 | RE | Failed | RLS/ | NAC/ketamine | RLS better |
| | | laminecotomy | pain | | increased activity/+/− pain relief |
| 7 | DE | Spinal | RLS/ | NAC/ketamine/ | RLS better |
| | | Stenosis | pain | methadone | trial to see if pain improves |
| | | | | | with addn NAC/Jan. 20, 2005) |
| 8 | ML | Spinal | RLS/ | NAC/ketamine | RLS better |
| | | Stenosis | pain | | RLS better on days when NAC |
| | | | | | used |
| 9 | Mw | Failed | pain | NAC/methadone | less pain |
| | | laminecotomy | | | more alert and less stammering |
| | | | | | with |
| | | | | | addition of NAC to methadone |

TABLE 3
Levels of enantiomers (ng/ml) of methadone (rmtd, smtd) and metabolite EDDP
(reddp, seddp), pain (yes/no; fatigue-Piper score) with and without NAC treatment

| # | dose | Rmtd | Smtd | reddp | Seddp | Fatigue | Med 1 | Med 2 | Med 3 |
|---|---|---|---|---|---|---|---|---|---|
| M001 | NAC 40 | 79.1 | 91.5 | 11.5 | 13.4 | No | Dyazide | Zyprexa 5 | Neurontin |
| | | | | | | (95) | | | 1200 |
| | NAC 40 | 80.4 | 89.6 | 12.2 | 89.6 | Yes | Dyazide | Zyprexa 5 | Neurontin |
| | | | | | | (197) | | | 1200 |
| M002 | NAC 30 | 48.9 | 49.2 | 8.9 | 11.3 | No(145) | Ketamine | Trazodone | |
| | | | | | | | 75 | 150 | |
| | NAC 30 | 41.2 | 36.4 | 9.3 | 11.7 | Yes | Ketamine | Trazodone | |
| | | | | | | (154) | 75 | 150 | |
| M003 | NAC | 124.7 | 124.9 | 11.9 | 13.8 | No(140) | Paxil 25 | Oxycodone | Lisinopril 5 |
| | 160 | | | | | | | 120 | |
| | NAC | 245.6 | 265.9 | 42.1 | 31.7 | No | Paxil 25 | Oxycodone | Lisinopril 5 |
| | 160 | | | | | (147) | | 120 | |
| M004 | Mtd | X | X | X | X | Yes | Ketamine | Oxycodone | Prozac 40 |
| | 160 | | | | | (210) | 200 | 60 | |
| | NAC | X | X | X | X | No | Ketamine | Oxycodone | Prozac 40 |
| | 160 | | | | | (121) | 200 | 60 | |
| M005 | Mtd 30 | X | X | X | X | Yes | Tramadol | | |
| | | | | | | (121) | 100 | | |
| | NAC 30 | X | X | X | X | No | Tramadol | | |
| | | | | | | (99) | 100 | | |
| M006 | Mtd 20 | X | X | X | X | Yes | Oxycodone | Prozac 60 | Tamoxifen |
| | | | | | | (234) | 60 | | 20 |
| | NAC 30 | X | X | X | X | No | Oxycodone | Oxycodone | Tamoxifen |
| | | | | | | (63) | 60 | 60 | 20 |

TABLE 4
Improvement in pain, clinical information and decrease in fraction of inactive
metabolite (reddp/reddp + seddp) with NAC treated in pain patients in clinical trial

| | Smoker/ | | VAS* | | | |
|---|---|---|---|---|---|---|
| Wt-sex | yr | Comments | (pain) | % r/r + s*** | Identifier | Comments |
| m-170 | y/42 | Inc. pain | 40% | 46.30% | M001 | Nerve injury left groin |
| | | | 80% | 47.30% | | |
| m-245 | n/44 | Inc.pain/w | 50% | 49.80% | M002 | Nerve injury sternum |
| | | | 70% | 53% | | |
| m-190 | y/51 | Inc. irritabil | 60% | 33% | M003** | Failed back surgery back pain |
| | | | 60% | 48% | | |
| f-250 | n/56 | Tired no | 70% | pending | M004 | Failed back surgery 2x leg pain |
| | | fatigue | 0% | | | Stopped all oxycondone |
| | | | (NAC) | | | subsequently; 30% opiod |
| | | | | | | reduction |
| m-245 | n/45 | | 0% | pending | M005 | Neck pain spinal stenosis |
| | | | 0% | | | “much less fatigue since starting |
| | | | (NAC) | | | NAC” |
| f-176 | n/50 | | 80% | Pending | M006 | Foot pain failed laminectory |
| | | | 30% | | | with meningomyelocoele “I feel |
| | | | (NAC) | | | like Iam walking around in a |
| | | | | | | daze” |
| | | | | | | “I do feel better taking NAC” |
| *Pain is shown by higher percentage | | | | | | |
| **Special attention to patient 3 who on high doses of methadone showed the greatest change with NAC therapy. He was taking paroxetine(paxil) a potent inhibitor of cyp2d6 which is essential in the metabolism of methadone and paxil. This patient noted increased irritability once stopping NAC as well as some change in his pain. He required paroxetine for control of a problem with anger. Paroxetine like methadone has an active metabolite and itself inhibits cyp2d6; | | | | | | |
| # while levels were not measured it is likely that his behavioral change off NAC was related to lower levels of the paroxetine metabolite. The patient continues to take NAC and is doing well. | | | | | | |

***fraction of inactive metabolite - While not measured, it is possible that, based on Kreek's work, that the presence of lower levels of inactive metabolite implies higher level of active metabolite accounting for improvement of fatigue scores and in some patients relief of pain.
+ Increased Pain
# Increased Irritability

Clinical Use of the Present Invention

Subjects to whom oral ketamine and NAC were administered have experienced relief from the symptoms of Restless Legs Syndrome (RLS). The blood levels of these patients measured below detection thresholds (20 ng/ml) and well below the blood levels recorded for pain relief (300 ng/ml). The NAC dose used in the present invention was far less than the 500 mg TID dose used as a supplement in treatment of diseases of inflammation.

### CASE HISTORIES

Case History No. 1.

A female patient with severe peripheral neuropathy treated with ketamine and methadone for three years noted increasing pain despite dose escalation of ketamine to 150 mg/day. The patient's leg pain increased despite control of her restless leg syndrome. The patient maintained a diary noting her level of pain with 1 being the lowest and 10 the highest. At the time, patient described her level of pain as 6-8. The patient was then administered 25 mg of NAC to be taken in combination with oral ketamine. Pain diary entries by the patient show a significant decrease in pain within 24 hours of administering NAC in combination with oral ketamine with the patient describing her pain at that time as a level 2 to 3. The patient continued treatment for two weeks, maintained the benefits of alleviated pain and reported no side effects. (The patient has continued on NAC with continued benefit during the subsequent year though she is now taking 1200 mg/day.)

Case History No. 2.

A morbidly obese female patient who had twice failed laminectomy surgery who was suffering from severe sleep apnea and severe leg pain was treated with a combination of tramadol and oral ketamine after a nearly fatal overdose of fentanyl which had been prescribed for her leg pain. The patient was administered ketamine for restless leg syndrome and noted pain relief at doses of 150 mg/day. Over a period of six months the level of pain relief diminished. Patient was subsequently administered NAC in combination with oral ketamine and noted that her pain lessened by 20-30% as compared with the combination of tramadol and oral ketamine. After one week of taking NAC patient noted no side effects and specifically no hallucinations or dissociative events.

Case History No. 3.

A male patient with restless legs syndrome who had failed back surgery noted an increase in pain after taking ketamine for restless leg syndrome for six months. The patient was then treated with a combination of NAC with oral ketamine and reported improvement in his restless leg syndrome and improved quality of sleep with his sleep duration increasing by two hours. The patient reported a 50% increase in his daily activity. In patient's pain diary, patient noted a pain score of 2-3 on a scale of 1-10 with 10 being the highest. No adverse effects were noted.

Treatment of a subject with the combination may be monitored using methods known in the art. The efficacy of treatment using the combination is preferably evaluated by examining the subject's symptoms in a quantitative way, e.g., by noting a decrease in the frequency of relapses, or an increase in the time for sustained worsening of symptoms. In a successful treatment, the subject's status will have improved (i.e., frequency of relapses will have decreased, or the time to sustained progression will have increased).

The invention now being fully described, it will be apparent to one of ordinary skill in the art that many changes and modifications can be made thereto without departing from the spirit or scope of the invention as set forth herein.

What is claimed is:

1: A composition for the treatment of pain or inflammation comprising N-acetyl-cysteine (NAC) or a derivative thereof and a pain or anti-inflammatory medication whose primary metabolism is modifiable by action of the cytochrome p450 system.

2: The composition of claim 1, wherein the pain medication is N-methyl-D-aspartate (NMDA) receptor antagonist.

3: The composition of claim 2, wherein the N-methyl-D-aspartate
(NMDA) receptor antagonist is selected from the group consisting of ketamine, dextromethorphan, memantine, amantadine, meperidine, methadone and mixtures and salts thereof.

4: The composition of claim 3, wherein the ketamine is oral ketamine.

5: The composition of claim 2, wherein the N-methyl-D-aspartate (NMDA) receptor antagonist is magnesium salts.

6: The composition of claim 1 the pain or anti-inflammation medication is a compound metabolized by the cytochrome P450 system into active form.

7: The composition of claim 1, wherein the pain or anti-inflammatory medication is selected from one or more steroids, non-steroid anti-inflammatory drugs, NSAIDs, essential fatty acids or fish oil products.

8: The composition of claim 7, wherein the essential fatty acids or fish oil products is omega-3 fatty acids.

9. The composition of claim 1, wherein the pain or anti-inflammatory medication is Resolvin, an active metabolite of omega-3-fatty acids made by the cyp450 system that may affect inflammation and pain.

10. The composition of claim 1, wherein the pain or anti-inflammatory medication is selected from morphine, methadone, oxycodone, hydromorphone, codeine, fentanyl or mixtures thereof.

11: The composition of claim 1, wherein the pain or anti-inflammatory medication is selected from oxycodone, prozac, dyazide, zyprexa, neurontin, trazodone, paxil or mixtures thereof.

12: The composition of claim 1, wherein the n-acetyl cysteine is present in amounts which increase the efficacy of the cytochrome P450 system in the production of active or inactive metabolites or engodgenous anti-inflammatory compounds.

13: The composition of claim 1, wherein the pain or anti-inflammatory medication is formulated as a time release, extended release, controlled release or sustained release formulary.

14: A kit for the treatment of pain or inflammation comprising the components of the composition of claim 1, wherein NAC or derivatives thereof and pain or anti-inflammatory medication are either separately present in individual containers or are together in the same container in relative amounts to treat pain or inflammation.

15. A method for the prophylaxis or treatment of pain and/or inflammation, comprising administering to a subject in need thereof the composition of claim 1.

16: A method for the prophylaxis or treatment of pain and/or inflammation, comprising administering to a subject in need thereof N-acetyl-cysteine (NAC) or derivatives thereof and pain or anti-inflammatory medication in relative amounts to provide prophylaxis or treatment of pain and/or inflammation.

17. The method of claim 15, wherein the subject has pain or inflammation associated with one or more of the following conditions including arthritis, neuropathic pain, multiple sclerosis, restless legs syndrome, sepsis, fibromyalgia, spinal stenosis, post surgical pain, post-laminectomy pain syndrome, post-thoracotomy pain syndrome, post-mastectomy pain syndrome, somatic pain, visceral pain, conditions characterized by pain and inflammation or neuro-inflammation like neuropathic pain, complex regional pain syndrome, or neuro-inflammatory conditions characterized by distressing sensations or condition characterized by neuroinflammation of the central nervous system.

18: The method of claim 17, wherein the subject is suspected of having a disease or condition selected from neuropathic pain, somatic pain, visceral pain and conditions associated with pain and neuro-inflammation.

19: The method of claim 16, wherein NAC or derivatives thereof and the pain or inflammation medication are administered separately.

20: The method of claim 16, wherein the NAC or derivatives thereof and the pain or inflammation medication are administered contemporaneously or sequentially.

21: The method of claim 15, wherein the pain or inflammation medication comprises one or more analgesic substances selected from non-steroidal anti-inflammatory drugs or essential fatty acids.

22: The method of claim 15, wherein the pain or inflammation medication is selected from opioids, methadone, morphine and tramadol.

23: The method of claim 15, wherein the pain or inflammation medication is N-methyl-D-aspartate (NMDA) receptor antagonist.

24: The method of claim 23, wherein the NMDA receptor antagonist is selected from the group consisting of ketamine, dextromethorphan, memantine, amantadine, meperidine, methadone and mixtures and salts thereof.

25: The method of claim 24, wherein the ketamine is oral ketamine.

26: The method of claim 23, wherein the N-methyl-D-aspartate (NMDA) receptor antagonist comprises magnesium salts.

27: The method of claim 15, wherein the pain or anti-inflammatory medication is selected from oxycodone, prozac, dyazide, zyprexa, neurontin, trazodone, paxil or mixtures thereof.

28: The method of claim 15, wherein the pain or anti-inflammatory medication is Resolvin, an active metabolite of omega-3-fatty acids made by the cyp450 system that may affect inflammation and pain.

29: The method of claim 15, wherein the pain or anti-inflammatory medication is formulate as a time release, extended release, controlled release or sustained release formulary.

30. A method of reducing the amount of an agent(s) being administered to a subject, comprising administering to a subject having a condition in need of treatment an effective amount of N-acetyl-cysteine (NAC) or derivatives thereof and a lesser than normal dosage amount of pain or inflammation medication ascribed for treatment of the condition, wherein the pain or inflammation medication is one that is metabolized in vivo to one or more active metabolites or endogenous product that decreases pain or inflammation by cytochrome P450 enzyme system or decreases the presence of metabolic products with toxicity.
